(19) United States
(12) Patent Application Publication
(10) Pub. No.: US 2007/0219458 A1
(43) Pub. Date: Sep. 20, 2007

(54) METHOD AND SYSTEM FOR DETERMINING HEARING STATUS

(75) Inventor: Patricia S. Jeng, Mahomet, IL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Mimosa Acoustics, Inc., Champaign, IL (US)

(21) Appl. No.: 11/624,032

(22) Filed: Jan. 17, 2007

Related U.S. Application Data
(60) Provisional application No. 60/759,831, filed on Jan. 17, 2006.

(51) Int. Cl. A61B 5/00; A61B 5/12

(52) U.S. Cl. 600/559; 73/585

(57) ABSTRACT

Method and system for determining hearing status. According to an embodiment, the present invention provides a method for ear screening. The method includes providing a probe that includes at least two output channels and an input channel. The method also includes determining a plurality of probe parameters for the probe. The plurality of parameters is related to at least a frequency response associated with the probe. In addition, the method includes performing a power measurement that includes at least outputting a first wide band stimulus from the two output channels of the probe to an ear canal. The method further includes obtaining a plurality of power parameters associated from the power measurement. The plurality of power parameters includes at least a pressure frequency response and an acoustic reflectance.

### CROSS-REFERENCES TO RELATED APPLICATIONS

This application claims priority to U.S. Provisional Application No. 60/759,831 filed Jan. 17, 2006, which is incorporated by reference herein.

### STATEMENT AS TO RIGHTS TO INVENTIONS MADE UNDER FEDERALLY SPONSORED RESEARCH OR DEVELOPMENT

Work described herein has been supported, at least in part, by Grant No. R44 DC006554 awarded by the Small Business Innovation Research Program. The United States Government may therefore have certain rights in the invention.

### BACKGROUND OF THE INVENTION

The present invention relates generally to screening techniques. More specifically, the invention provides a method and system for screening inner and middle ears in an integrated process. As an example, a specific embodiment of the present invention utilizes both wideband reflectance based technique and/or intensity calibration in the process of screening hearing problems. In a specific embodiment, the present invention is effective for screening hearing disorders for infants and young children. Adults and the elderly are also subject to many inner and middle ear problems. This system is applicable to this group as well. Merely by way of an example, the invention has been applied to screening hearing problems, but it would be recognized that the invention has a much broader range of applicability.

Hearing, is an important aspect of life for every human being, critical to a social well-being. Unfortunately, many people lose their ability to hear over the course of the lives, or are born without hearing. Fortunately, if caught early, hearing losses and/or impairments in many instances may be mitagated, or even cured. A necessary step in mitagating and/or curing hearing problems involves proper early diagnosis and screening to determine what causes hearing problems.

Over the past, various techniques have been developed for screening hearing problems. Almost twenty years ago, a scientist named David Kemp in 1978 demonstrated the existence of otoacoustic emission (OAE), a sound that is generated from within the inner ear. Since then, various techniques have evolved around the OAE concept. For example, two methodologies—transient OAE (TOAE) and distortion product OAE (DPOAE)—have been developed using the OAE concept. Commonly, especially in Europe, TOAE is evoked using stimulus characterized by a broad frequency range, with DPOAE being more popular in the US markets. Both evoked responses from these types of stimului may cover the frequency range up to around 4 kHz, however DPOAE typically goes much higher, even to 15 kHz. A DPOAE is evoked using stimuli that include a tone pair with predetermined intensity and frequency levels. The evoked response from these stimuli occurs at a third frequency. The distortion product frequency is then calculated based on the two original frequencies as the lower frequency, less the difference (upper-lower).

Over the past, OAE methodologies have been developed and used extensively for determining hearing disorders associated with inner ears. For example, used along with auditory brainstem responses (ABR) measurement systems, DPOAE and TEOAE are specifically aimed at detecting disfunctions in the outer hair cell (OHC) in the cochlear (also referred to as inner ear), while ABR gives general information about the overall functioning of the cochlear and auditory nerves up to the brainstem.

FIG. 1 is a simplified diagram illustrating a conventional DPOAE system. As shown in FIG. 1, a DPOAE system includes a user interface module 101, a calibration module 102, a measurement module 103, a data module 104, and a database module 105. In operation, the user interface module 101 provides user interface (e.g., computer monitor, input devices, etc.) for the operator. The calibration module 102 is used for calibration the DPOAE for performing measurement. For example, the calibration module 102 compares the newly acquired measurements against known measurements and makes adjustments on the DPOAE system accordingly. The measurement module 103 is used for performing actual measurements in a target ear canal. The data module 104 is used for providing various functions for processing data acquired during measurement processes. The database module 105 stores information related the measurement process. For example, database module 105 stores information for known measurements that is used during calibration processes.

Unfortunately, these measurements are often affected by the middle ear status, which can be blocked, limiting the signal from reaching the inner, and thereby reducing their reliabilities. For example, if the external ear canal and/or the middle ear are not functioning properly, the measurements of DPOAE, TEOAE, and ABR will likely provide false positive results.

To solve this problem, currently when these screening tests give a positive result, the tested ear has to be tested again using different instrument to evaluate the middle ear status in order to confirm the positive status (i.e., indication of hearing disorders) of the inner ear. For example, a common standard used for screening the middle ear is a single-frequency tympanometry at 226 Hz.

For example, the pressure range for screening products between −300 daPa to +200 daPa. There are usually 2 different ranges available on diagnostic products: −400 daPa to +200 daPa and −600 daPa to +400 daPa. As a reference, ANSI and IEC standards require a maximum limit protection at +800 daPa and −400 daPa. As an example, the pressure sweep is generally from positive to negative pressure and the sweep rates vary from as slow as 12.5 daPa/s to as fast as 600 daPa/s. Most clinics use only 226 Hz for testing anyone older than 6 months of age. If the patient is under 6 months, then they would use 1000 Hz.

Unfortunately, conventional techniques such as tympanometry have various serious limitation limitations. In a specific example, this technique does not work in infants under 6-month of age, as infants ears are usually much more compliant compared to adult ears, and are limited in its frequency range. The typical tympanometry test is done at 225 Hz, with some systems testing at 1 kHz. These higher frequency systems are still in the experimental stage, and are not yet known to give reliable diagnosis. In certain instances, the pressure sweep from −400 deca-pascal to +200 deca-pascal in the tympanometry does not give reliable results in this population. Worse yet, the follow up tests can take another few weeks to months (often due to rescheduling problems), or does not happen at all.

One way to ensure accuracy of tests, and thus greater reliability, for inner ears, and to solve the false positive problem due to middle ear transmission problems, has been to calibrate and test middle ears as well. Among these, Mimosa Acoustics, Inc. developed an array of instruments for solving these problems. As an example, Mimosa Acoustics, Inc. has developed an acoustic reflectance measurement system for measuring, among other things, middle ear disorders. It is to be appreciated that the use of acoustic reflectance is discussed in a related application, which is the U.S. patent application Ser. No. 11/061,368 filed on Feb. 18, 2005, which is herein incorporated by reference for all purposes.

FIG. 2 is a simplified diagram illustrating a conventional reflectance measurement system. A reflectance measurement system 200 includes a user interface module 201, a calibration module 202, a measurement module 203, a data module 204, a database module 205, and a computation module 206. In operation, the user interface module 201 provides user interface (e.g., computer monitor, input devices, etc.) for the operator. The calibration module 202 is used for calibration the reflectance measurement system 200 for performing measurement. For example, the calibration module 202 measures a set of values for a reference cavity, and then the calibration module 202 compares the measure set of values against known reference values associated with the cavity. Based on this comparison, properly adjustment is then made. The measurement module 203 is used for performing actual measurements in a target ear canal. The incidental pressures and acoustic frequencies are measured. The computation module 206 is used to determine an acoustic reflectance value based on the incidental pressures and acoustic frequencies. The data module 204 is used for providing various functions for processing data acquired during measurement processes. The database module 205 stores information related the measurement process. For example, database module 205 stores information for known measurements that is used during calibration processes.

The reflectance measurement system as described above provides a certain level of solution for screening hearing disorders. Unfortunately, using reflectance measurement system alone is often inadequate.

Therefore, it is desirable to have a method and system for reliable screen hearing disorders.

### BRIEF SUMMARY OF THE INVENTION

The present invention relates generally to screening techniques. More specifically, the invention provides a method and system for screening inner and middle ears in an integrated process. As an example, a specific embodiment of the present invention utilizes both wideband intensity reflectance/transmittance based technique and/or intensity calibration in the process of screening hearing problems. In a specific embodiment, the present invention is effective for screening hearing disorders for both infants and young children, as well as the adult and elderly populations. Merely by way of an example, the invention has been applied to screening hearing problems, but it would be recognized that the invention has a much broader range of applicability.

According to an embodiment, the present invention provides a method for ear screening. The method includes providing a probe that includes one, two or more output channels and at least one input channel. The method also includes determining a plurality of probe parameters for the probe. The plurality of parameters is related to at least a frequency pressure or velocity response associated with the probe, as well as the source impedance or admittance of the probe. In addition, the method includes performing a power (intensity) measurement, that typically includes outputting a first wide band stimulus from the two output channels of the probe to an ear canal. The method further includes obtaining a plurality of power parameters associated from the power measurement. The plurality of power parameters includes at least a pressure frequency response and an acoustic reflectance. The method additionally includes determining an output level that is associated with the pressure frequency response. The method also includes performing an otoacoustic emission measurement using the output level.

According to another embodiment, the present invention provides a method for ear screening. The method includes providing a probe that includes at least two output channels and an input channel. The method also includes calibrating a plurality of probe parameters for the probe. For example, the plurality of parameters is related to at least a frequency response associated with the probe. The method additionally includes outputting a wide band stimulus from the two output channels. The method also includes measuring a first plurality of power parameter responses. For example, the first plurality of power parameter responses are associated with the first wide band stimulus. The first plurality of power parameter responses includes at least a pressure frequency response and an acoustic reflectance. Furthermore, the method includes outputting a first pair of pure tones from the two output channels. The method additionally includes measuring a second plurality of power parameter responses is associated with the first pair of pure tones. The method further includes determining a first output level and a second output level. The first output level and the second output level are associated with the pressure frequency response. The method also includes outputting a second pair of pure tones that includes a first pure tone and a second pure tone. The first pure tone is associated with the first output level. The second pure tone is associated with the second output level. The method additionally includes determining a distortion product otoacoustic emission response that is associated with the second pair of pure tones.

According to yet another embodiment, the present invention provides a system for ear screening. The system includes a processor module. The system also includes a memory module. The system additionally includes a calibration module that includes at least a cavity set. For example, the cavity set is characterized by a plurality of known cavity parameters. The system further includes a probe module that includes at least two output channels and an input channel. In addition, the system includes a connection module that is coupled to the processor module and the probe module. The probe module is configure to generate a first stimulus using the at least two output channels and determine a first plurality of measurements. The first plurality of measurement includes at least a reflectance measurement. The probe is further configure to generate a second stimulus using the at least two output channels. The second stimulus is characterized by a output level that is associated with the reflectance level. The probe is further configured to determine a second plurality of measurements that is associated the second stimulus.

It is to be appreciated that various embodiments of the present invention offers numerous advantages over conventional techniques. Certain embodiments of the present invention performing screening for both middle and inner ear problems using an integrated system, and the screening process is performed in a single session. For example, a reflectance based system is used for detecting middle ear problems and an OAE based system is used for detecting inner ear problems. In addition, various embodiments of the present invention provide mechanisms to ensure reliability of OAE based measurements. In addition, various embodiments of the present invention are compatible with conventional techniques and easily implemented. For example, a system according to embodiments of the present invention may be implemented using existing hardware tools. There are other benefits as well.

Depending upon embodiment, one or more of these benefits may be achieved. These benefits and various additional objects, features and advantages of the present invention can be fully appreciated with reference to the detailed description and accompanying drawings that follow.

### BRIEF DESCRIPTION OF THE DRAWINGS

FIG. 1 is a simplified diagram illustrating a conventional DPOAE system.

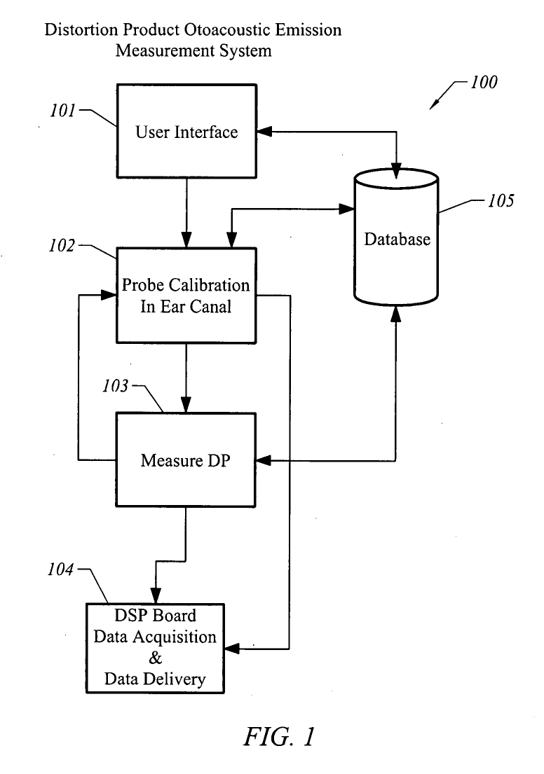

FIG. 2 is a simplified diagram illustrating a conventional reflectance measurement system.

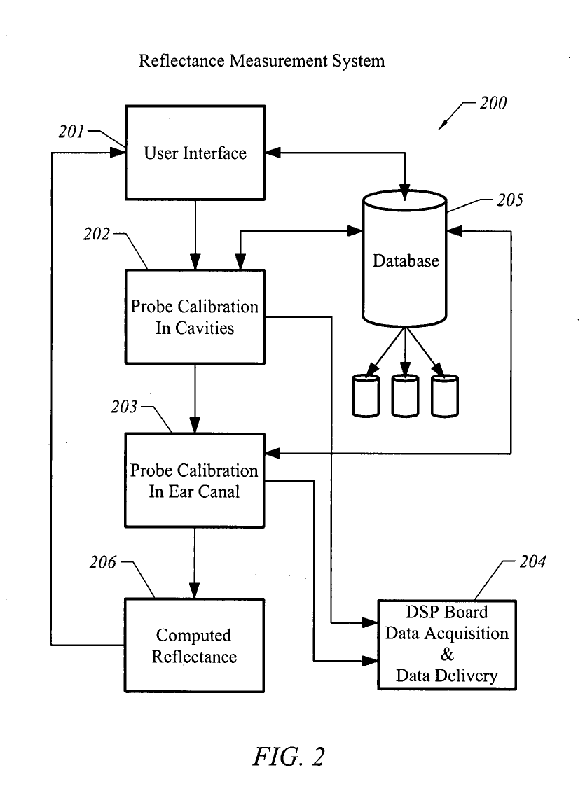

FIG. 3 is a simplified diagram illustrating a system for diagnosing hearing disorders according to an embodiment of the present invention.

FIG. 4 is a simplified block diagram of software modules according to an embodiment of the present invention.

FIG. 5 is a simplified diagram illustrating the process for performing measurements according to an embodiment of present invention.

### DETAILED DESCRIPTION OF THE INVENTION

The present invention relates generally to screening techniques. More specifically, the invention provides a method and system for screening inner and middle ears in an integrated process. As an example, a specific embodiment of the present invention utilizes both wideband reflectance based technique and/or intensity calibration in the process of screening hearing problems. In specific embodiments, the present invention is effective for screening hearing disorders for infants and young children. Merely by way of an example, the invention has been applied to screening hearing problems, but it would be recognized that the invention has a much broader range of applicability.

As described, conventional techniques are often inadequate for thorough diagnosis of hearing related problems. More specifically, conventional techniques fail to reliably detect both middle and inner ear problems, and diagnosis of inner ear problem is often affected by the middle ear problems. It is to be appreciated that embodiments of the present invention offers an integrated solution for reliably detecting both middle and inner ear problems.

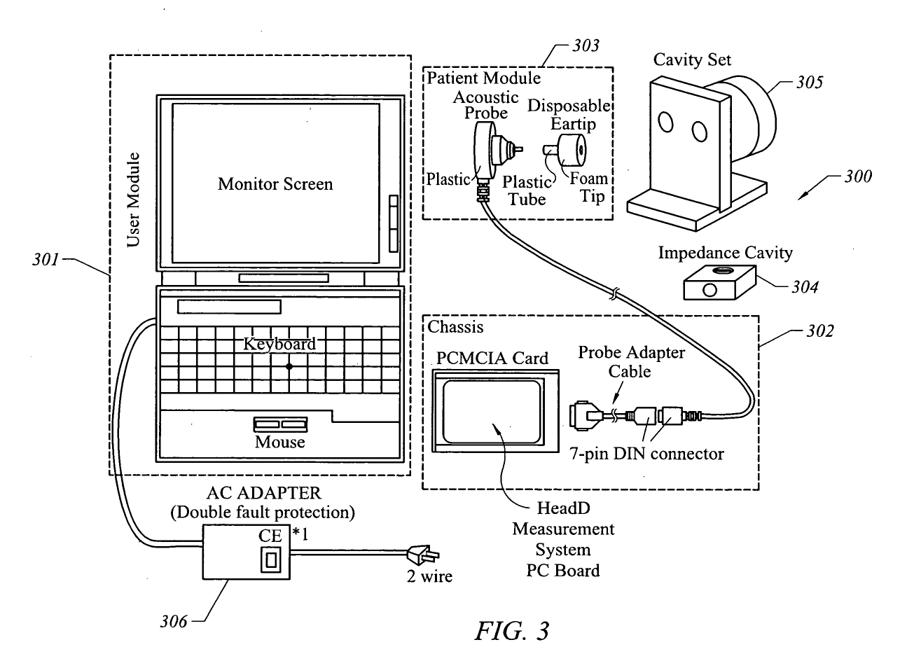

FIG. 3 is a simplified diagram illustrating a system for diagnosing hearing disorders according to an embodiment of the present invention. This diagram is merely an example, which should not unduly limit the scope of the claims. One of ordinary skill in the art would recognize many variations, alternatives, and modifications. For the purpose of illustration, the system is capable of performing both OAE and MEPA measurements. But it is understood the system may perform other measurements as well.

As shown in FIG. 3, the system 300 includes the following components:

1. a user module 301;

2. a connector module 302;

3. a patient module 303;

4. an impedance cavity 304; and

5. a cavity set 305.

The test signals are generated by the user modules 301 (e.g., personal computer, PDA, etc.) and delivered through the connector module 302 (e.g., connectors, cables, etc.) to the patient module 303. For example, the patient module 303 includes an acoustic probe. A microphone within the acoustic probe is able to pick up external signals and transmits the electrical signal through the connector module 302 to the user module 301 for recording and processing.

In an embodiment, the acoustic probe houses two output channels (e.g., two speaker transducers) and one input channel (e.g., one microphone transducer). In comparison, as a stand-alone system, only one output and one input channels are required for MEPA, and two output and one input channels are required for performing DPOAE measurement. According to embodiments of the present invention, two output channels are used for both measurements. In a specific embodiment, the MEPA is measured using both channels, while DPOAE is measured once.

The impedance cavity 304 is used as an ear simulator for calibration purposes. For example, the impedance cavity 304 is used before each measurement in place of the probe calibration in the cavity set. As another example, the impedance cavity 304 is used as independent check of the probe integrity. There are other functions as well.

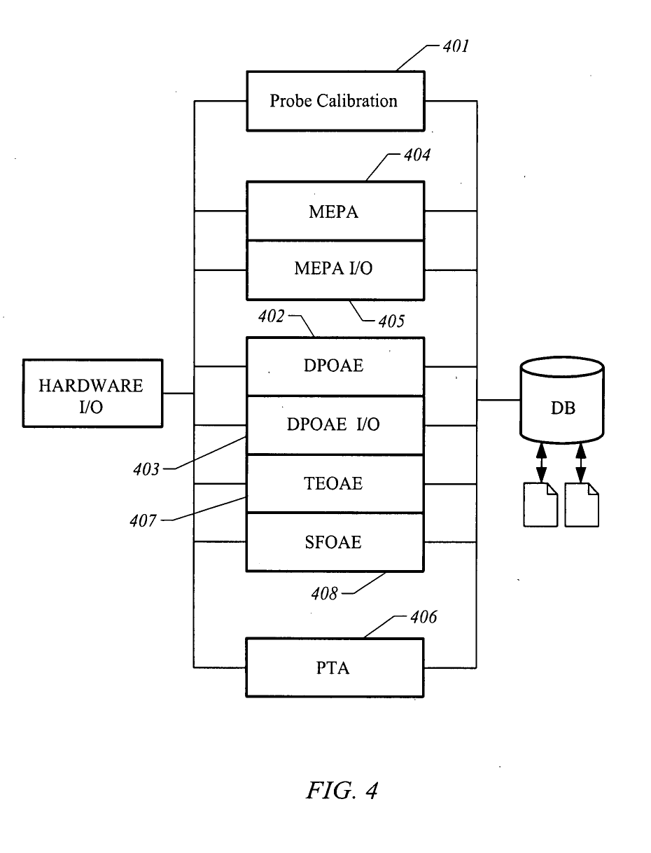

The system 300 is operated in conjunction with software modules. For example, software modules loaded on the user module 301. FIG. 4 is a simplified block diagram of software modules according to an embodiment of the present invention. This diagram is merely an example, which should not unduly limit the scope of the claims. One of ordinary skill in the art would recognize many variations, alternatives, and modifications.

As shown, the software modules include the following:

1. a probe calibration module 401;

2. a DPOAE Module 402;

3. a DPOAE I/O-function Module 403;

4. an MEPA Module 404;

5. an MEPA I/O-function Module 405;

6. a PTA Module 406;

7. a TEOAE Module 407; and

8. an SFOAE Module 408.

According to an embodiment, the probe calibration module 401 performs various functions related to calibration of the system. In certain embodiments, the probe calibration module is use specifically for performing calibration using a reference cavity set, which are For example, the probe calibration module performs certain aspects of sound probe calibration, which may provide the followings:

1. The factory determined probe sensitivity for speaker and microphone with equalization based on an artificial ear reference;

2. The in-the-ear sound pressure calibration either with a pure tone at 1 kHz and/or a wide band (e.g., chirp) response;

3. The Thevenin parameter calibration from stored data and/or from cavity response measurements; and

4. The new sound intensity calibration building on in-the-ear sound pressure calibration and probe Thevenin parameter data. For example, the parameters are independent from artificial ear reference.

It is to be understood that the calibration module 401 may perform other calibration functions as well.

The MEPA module 404 is used for performance various reflectance measurements. For example, the MEPA Module 404 performs ear canal reflectance and/or impedance measurement using various types of stimuli, such as wide band, sine, and/or multi-sine stimuli. In certain embodiments, sound pressure calibration of the stimuli is performed. Alternatively, sound intensity calibration of the stimuli is performed. In certain embodiments, various measurements (e.g., reflectance, pressure, etc.) determined by the MEPA module 404 are used for the calibration of OAE type system and/or methods.

The MEPA I/O-function module 405 is used for provide reflectance I/O-functions. For example, reflectance I/O functions include calibration for sound pressure, intensity. In addition, the reflectance I/O functions may include providing wide band, sine, and/or multi-sine stimuli. In certain embodiments, heterodyne method is used for providing sine stimulus. In certain embodiments, the MEPA I/O function module 405 is an integral part of the MEPA module 404.

The DPOAE module 402 is used for providing various DPOAE related measurements, whose detailed operation is provided below. In various embodiments, the DPOAE module 402 is calibrated using a reflectance value determined by the MEPA module 404.

The DPOAE I/O-function module 403 is used for provided various I/O functions for the purpose of performing measurements. More specifically, the I/O functions include providing configurable sound output levels (e.g., pressure level, intensity level, etc.), which may be achieved by changing output voltage levels that are used for generating tones used during measurement processes. In a specific embodiment, the I/O functions include providing configurable frequency levels and/or intensity levels.

In addition, various characteristics of the DPOAE measures may be determined by the DPOAE I/O-function module 403. For example, the DP-threshold estimation may be determined by performing extrapolation of DP-I/O-functions. As another example, heterodyne method may also be used. In certain embodiments, the DPOAE I/O-function module 403 is an integral part of the DPOAE module 402.

The TEOAE module 407 is used for providing TEOAE measurement with sound pressure and intensity calibration. In certain embodiment, spectrum calibration is also performed. For example, spectrum calibration involves compensating stimulus spectrum from ear-canal responses with respect to pressure or intensity calibration. In various embodiments, the DPOAE module 402 is calibrated using a reflectance value determined by the MEPA module 404. In various embodiments, the TEOAE module 402 is calibrated using a reflectance value determined by the MEPA module 404.

The SFOAE module 408 is used for providing SFOAE measurement with sound pressure and/or intensity calibration. In various embodiments, the SFOAE module 402 is calibrated using a reflectance value determined by the MEPA module 404.

The PTA module 406 is used for providing pure-tone audiogram with sound pressure and/or intensity calibration. In a specific embodiment, the sound pressure calibration is in compliance with the ANSI standard.

It is to be appreciated that depending upon specific applications, various modules may be added, removed, combined, and/or replaced based on specific implementation needs.

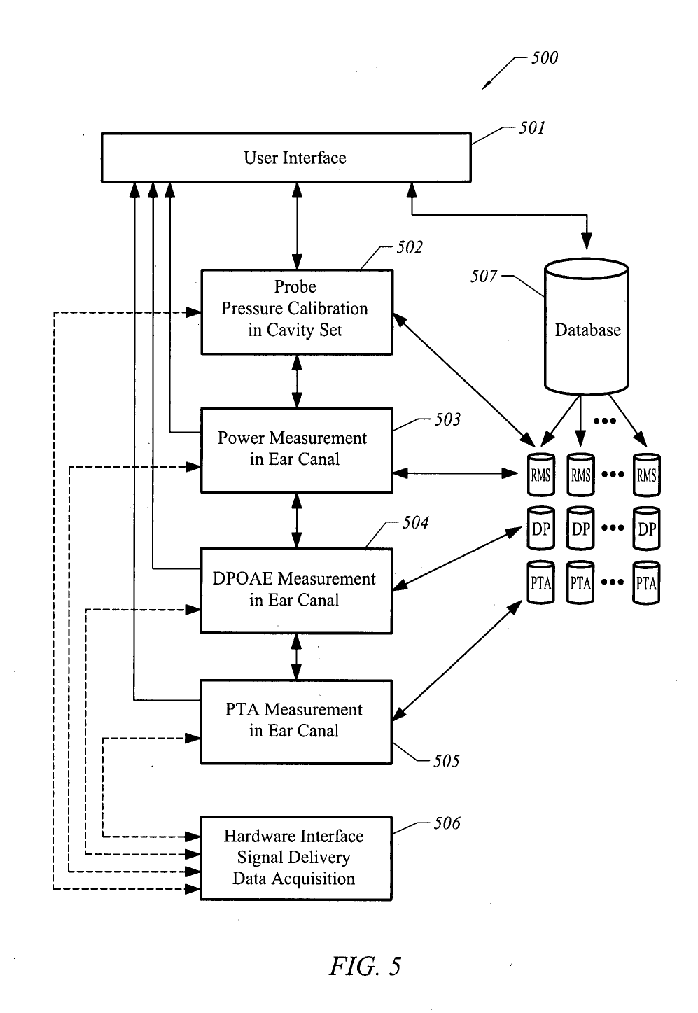

FIG. 5 is a simplified diagram illustrating the process for performing measurements according to an embodiment of present invention. This diagram is merely an example, which should not unduly limit the scope of the claims. One of ordinary skill in the art would recognize many variations, alternatives, and modifications. For example, various steps as illustrated may bed added, removed, repeated, replaced, rearranged, overlapped, and/or partially overlapped.

As shown in FIG. 5, the following processes are performed:

1. calibration 502;

2. power measurement 503;

3. OAE measurement 504; and

4. PTA measurements 505.

Each of the above processes is performed using software and hard components. As an example, the above processes are performed by the system 300 as shown in FIG. 3. For example, the user interface 501 as shown allows a user or operator to control these processes. For example, the user interface 501 includes a display, and keyboard, and a mouse. The hardware interface 506 is provided so that various processes as listed above may function. For example, the hardware interface 506 includes connections to a probe set that is used for performing various measurements in ear canals. The database 507 is used to store various information related to performing measurements in ear canals. For example, the database 507 stores data related to predetermined reference data. In addition, the database 507 may also store information acquired during the measuring process.

According to certain embodiments, the calibration 502 is performed before other processes. Depending upon the equipment used, various types of calibration may be necessary. In a specific embodiment, probe pressure calibration is performed using a cavity set. First, Thevenin equivalent parameters for the probe output transducers are determined. For example, the Thevenin equivalent parameters are obtained by measuring the multiple pressure frequency responses of the probe in the cavities of the cavity set. As an example, the cavity set includes model ear tips in various sizes. The frequency responses are then used to estimate the Thevenin equivalent parameters of the ear probe.

In addition, to ensure the reliability of the calibration process, the reflectance of the source transducer is compared to the known reference reflectance (e.g., predetermined values stored in the database 507) of the reference cavity set. For example, if more than 90% of the measured reflectance over the frequency range falls within a range of the reference reflectance value, the measured Thevenin equivalent parameters are determined as valid. Otherwise, the calibration process is repeated until the probe set is properly calibrated. Depending on the specification, other reference value and threshold levels may be used.

Next, power measurement 503 is performed. In an embodiment, the power measurement is performed in an ear canal using a probe set. For example, the probe set as shown in FIG. 3 is used and includes transducers, thus providing two output channels and a single input channel.

Typically, to ensure the accuracy and reliability of the power measurement, especially the frequency response measurements, precaution is taken in various ways:

1. check various connections, especially connection to the probe set;

2. check the sealing and/or contact between the probe and the ear canal;

3. check to make sure that the transducer port portion of the probe is not positioned against any walls of the ear call; and

4. check to entry port portion of the transducers is not clogged by the ear canal wax and/or vernex.

There might be other checks performed before power measurement process is performed. Once these checks are performed, power measurements are then performed. In a specific embodiment, the power measurement process involves measuring the pressure frequency response of the probe in the tested ear canal for both output channels using a wide band stimulus. It is to be understood that other types of stimuli may be used as well. As an example, the wide band (e.g., chirp) stimulus is often useful for power measurements, as a wide band stimulus gives provide a wideband stimulus (i.e., a number of frequency points). In comparison, pure tone stimuli provide fewer numbers of frequencies points, but are less susceptible to background noises.

Based on the frequency responses, various power parameters and determined. In a specific embodiment, the newly determined power parameters are display on the user interface 501. For example, these parameters include the ear power reflectance, reflectance phase, reflectance group delay, power absorption, transmittance, normalized impedance (e.g., including magnitude, phase, group delay, resistance, and reactance), impedance in MKS, admittance (including magnitude, phase, group delay, conductance, and susceptance), sound pressure level, pressure group delay, the sound intensity level, equivalent volume, etc. It is to be understood that there might be other parameters as well.

Once power parameters associated with the wide band stimulus is determined, the power measurement process is performed again with pure tone stimuli. In an embodiment, pure tones are delivered simultaneously. Usually, at the low frequency region, the frequency responses obtained using wide band is susceptible to the background noises (which include external and internal noises). For example, the external noise may come from environment, such as equipment noise, rubbing of the probe cable against clothing, etc. The internal noise usually comes from physiological noises, such as blood flow in the ear canal or swallowing, coughing, sucking noise, etc.

After power measurements, OAE measurement 504 is performed. As merely an example, DPOAE measurement is illustrated in FIG. 5 and explained below. But it is to be understood that other types OAE measurements (e.g., TOAE, SFOAE, etc.) may be implemented according to embodiments of the present invention.

According to an embodiment, the DPOAE measurement is performed in an ear canal using a pair of pure tones and record the response from the ear canal the determine a cubit distortion product. As an example the pure tones are characterized by frequencies F₁ and F₂ and output levels L₁ and L₂. The pure tone pairs are presented to the ear canal for measuring from high frequency to low frequency. Depending upon the specific application, the output levels may be related to sound pressure level and/or sound intensity level. In certain embodiments of the present invention, it is advantageous to use sound intensity levels. For example, by using sound intensity levels, the OAE measurement is less susceptible to misrepresentation caused by standing wave at some primary tone frequencies.

Typically, sound wave problems are related to pressure waves in the ear canal. Usually, when no reflections are present there is no retrograde (i.e., reflected) pressure wave to interfere with the incident (i.e. forward moving) pressure wave. However, if there are reflections, as occurs with an acoustic impedance mismatch at the eardrum, the retrograde pressure wave interferes with the incident pressure wave, thereby causing a periodic sequence of peaks and valleys of pressure as the incident and retrograde waves go in and out of phase with each other. Under these conditions, the variation in sound pressure level (SPL) with distance in the ear canal can be substantial. For example, several studies have observed variations in SPL greater than 20 dB, at frequencies as low as 3.5 kHz.

Pressure measurements thus may produce misleading interpretations due to standing waves. Since standing waves vary substantially with frequency, the greater the bandwidth of the signal, the more confounding are the reflected waves. For example, in a DPOAE measurements involve 3 tones, each of which has its own standing wave pattern. As another example, a TEOAE measurement involves a broadband signal resulting in a complex variation of signal level across frequency due to the standing wave problem.

Therefore, it is to be appreciated that in performing OAE measurements, sound intensity levels are used. Typically, sound intensity (or sometimes referred as acoustic intensity) is defined as the power flow per cross-sectional area. Because power flows uniformly along the length of the ear canal with negligibly small losses at the walls of the ear canal due to friction, it is not subject to the standing wave problem. In order to get the intensity, the acoustic input impedance, another with other parameters, of the ear canal needs to be determined. According to various embodiments, these parameters may be determined during the power measurement process.

In addition, for the DPOAE measurements performed accurately and reliably, proper output levels output levels L₁ and L₂ are required. In conventional techniques, lengthy and tedious procedures are often required in order to determine the proper output levels output levels L₁ and L₂. In contrast, embodiments according to the present invention determines proper output levels based on pressure frequency response measurements obtained from the power measurement process described above.

After the OAE measurement is performed, pure tone audiometry (PTA) measurement 505 is performed. Typically, a PTA measurement is for measuring one's response to a pure tone audiogram and compare the measurements against audible hearing thresholds of the pure tones in patient's ear canal. For example, the measured hearing thresholds can be in forms of sound pressure levels (SPL) and/or sound intensity levels (SIL).

According to an embodiment, the present invention provides a method for ear screening. The method includes providing a probe that includes at least two output channels and an input channel. The method also includes determining a plurality of probe parameters for the probe. The plurality of parameters is related to at least a frequency response associated with the probe. In addition, the method includes performing a power measurement that includes at least outputting a first wide band stimulus from the two output channels of the probe to an ear canal. The method further includes obtaining a plurality of power parameters associated from the power measurement. The plurality of power parameters includes at least a pressure frequency response and an acoustic reflectance. The method additionally includes determining an output level that is associated with the pressure frequency response. The method also includes performing an otoacoustic emission measurement using the output level. For example, the embodiment is illustrated according to FIG. 5.

According to another embodiment, the present invention provides a method for ear screening. The method includes providing a probe that includes at least two output channels and an input channel. The method also includes calibrating a plurality of probe parameters for the probe. For example, the plurality of parameters is related to at least a frequency response associated with the probe. The method additionally includes outputting a wide band stimulus from the two output channels. The method also includes measuring a first plurality of power parameter responses. For example, the first plurality of power parameter responses are associated with the first wide band stimulus. The first plurality of power parameter responses includes at least a pressure frequency response and an acoustic reflectance. Furthermore, the method includes outputting a first pair of pure tones from the two output channels. The method additionally includes measuring a second plurality of power parameter responses is associated with the first pair of pure tones. The method further includes determining a first output level and a second output level. The first output level and the second output level are associated with the pressure frequency response. The method also includes outputting a second pair of pure tones that includes a first pure tone and a second pure tone. The first pure tone is associated with the first output level. The second pure tone is associated with the second output level. The method additionally includes determining a distortion product otoacoustic emission response that is associated with the second pair of pure tones. For example, the embodiment is illustrated according to FIG. 5.

According to yet another embodiment, the present invention provides a system for ear screening. The system includes a processor module. The system also includes a memory module. The system additionally includes a calibration module that includes at least a cavity set. For example, the cavity set is characterized by a plurality of known cavity parameters. The system further includes a probe module that includes at least two output channels and an input channel. In addition, the system includes a connection module that is coupled to the processor module and the probe module. The probe module is configure to generate a first stimulus using the at least two output channels and determine a first plurality of measurements. The first plurality of measurement includes at least a reflectance measurement. The probe is further configure to generate a second stimulus using the at least two output channels. The second stimulus is characterized by a output level that is associated with the reflectance level. The probe is further configured to determine a second plurality of measurements that is associated the second stimulus. For example, the embodiment is illustrated according to FIG. 3.

It is to be appreciated that various embodiments of the present invention offers numerous advantages over conventional techniques. Certain embodiments of the present invention performing screening for both middle and inner ear problems using an integrated system, and the screening process is performed in a single session. For example, a reflectance based system is used for detecting middle ear problems and an OAE based system is used for detecting inner ear problems. In addition, various embodiments of the present invention provide mechanisms to ensure reliability of OAE based measurements. In addition, various embodiments of the present invention are compatible with conventional techniques and easily implemented. For example, a system according to embodiments of the present invention may be implemented using existing hardware tools. There are other benefits as well.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for ear screening, the method comprising:
providing a probe, the probe including at least two output channels and an input channel;
determining a plurality of probe parameters for the probe, the plurality of parameters being related to at least a frequency response associated with the probe;
performing a power measurement, the power measurement including at least outputting a first wide band stimulus from the two output channels of the probe to an ear canal;
obtaining a plurality of power parameters associated from the power measurement, the plurality of power parameters including at least a pressure frequency response and an acoustic reflectance;
determining an output level, the output level being associated with the pressure frequency response; and
performing an otoacoustic emission measurement using the output level.

2. The method of claim 1 wherein the output level is further being associated with the acoustic reflectance.

3. The method of claim 1 wherein the performing an otoacoustic emission comprises:
providing a first tone and a second tone, the first one and the second tone are characterized by different frequency levels;
setting the first tone to a first sound level.

4. The method of claim 3 wherein the first sound level comprises a sound pressure level.

5. The method of claim 3 wherein the first sound level comprises a sound intensity level.

6. The method of claim 3 wherein the first sound level is associated with the acoustic reflectance and an impedance value.

7. The method of claim 1 there the acoustic reflectance is associated with the ear canal as a function of an incident pressure and an acoustic frequency.

8. The method of claim 1 further comprising performing pure tone audiometry.

9. The method of claim 1 wherein the power measurement further includes:
outputting pure tones; and
obtaining at a least low frequency response.

10. The method of claim 1 wherein the otoacoustic emission measurement comprises a distortion product otoacoustic emission measurement.

11. The method of claim 1 wherein the otoacoustic emission measurement comprises a transient otoacoustic emission measurement.

12. The method of claim 1 wherein the otoacoustic emission measurement comprises a stimulus frequency otoacoustic emission measurement.

13. The method of claim 1 further comprising sealing the probe to the ear canal.

14. The method of claim 1 further comprising comparing probe parameters to a set of predetermined parameters associated with a cavity set.

15. A method for ear screening, the method comprising:
providing a probe, the probe including at least two output channels and an input channel;
calibrating a plurality of probe parameters for the probe, the plurality of parameters being related to at least a frequency response associated with the probe;
outputting a wide band stimulus from the two output channels;
measuring a first plurality of power parameter responses, the first plurality of power parameter responses being associated with the first wide band stimulus, the first plurality of power parameter responses including at least a pressure frequency response and an acoustic reflectance;
outputting a first pair of pure tones from the two output channels;
measuring a second plurality of power parameter responses, the second plurality of power parameter responses being associated with the first pair of pure tones;
determining a first output level and a second output level, the first output level and the second output level being associated with the pressure frequency response;
outputting a second pair of pure tones, the second pair of pure tones including a first pure tone and a second pure tone, the first pure tone being associated with the first output level, the second pure tone being associated with the second output level; and
determining a distortion product otoacoustic emission response, the otoacoustic emission response being associated with the second pair of pure tones.

16. The method of claim 15 further comprising storing the first plurality of power parameter responses.

17. A system for ear screening, the system comprising:
a processor module;
a memory module;
a calibration module, the calibration module including at least a cavity set, the cavity set being characterized by a plurality of known cavity parameters;
a probe module, the probe module including at least two output channels and an input channel; and
a connection module, the connection module being coupled to the processor module and the probe module;
wherein:
the probe module is configure to generate a first stimulus using the at least two output channels and determine a first plurality of measurements, the first plurality of measurement including at least a reflectance measurement;
the probe is further configure to generate a second stimulus using the at least two output channels, the second stimulus being characterized by a output level, the output level being associated with the reflectance level;
the probe is further configured to determine a second plurality of measurements, the second plurality of measurements being associated the second stimulus and an otoacoustic emission.

18. The system of claim 17 wherein the processor module includes a computer.

19. The system of claim 17 wherein the memory module comprises a database.

20. The system of claim 17 wherein the first stimulus comprises a wide band stimulus.

21. The system of claim 17 wherein the first stimulus comprises a chirp stimulus.

22. The system of claim 17 wherein the first stimulus comprises a pure tone stimulus.
